(19) United States
(12) Patent Application Publication
(10) Pub. No.: US 2011/0218613 A1
(43) Pub. Date: Sep. 8, 2011

(54) Vascular Prosthesis Assembly with Retention Mechanism and Method

(75) Inventors: Eric W. Leopold, Redwood City, CA (US); Eric Hsiang Yu, Moraga, CA (US); Alexander Arthur Lubinski, Rocklin, CA (US); Matthew J. Wioncek, San Jose, CA (US)

(73) Assignee: NovoStent Corporation, Mountain View, CA (US)

(21) Appl. No.: 12/879,965

(22) Filed: Sep. 10, 2010

Related U.S. Application Data
(60) Provisional application No. 61/241,345, filed on Sep. 10, 2009.

(51) Int. Cl. A61F 2/84; A61F 2/82

(52) U.S. Cl. 623/1.12; 623/1.2

(57) ABSTRACT

A vascular prosthesis assembly includes a self expanding prosthesis and a selectively releasable retention mechanism over the outer surface of the prosthesis which maintains the vascular prosthesis in a contracted state. The retention mechanism may include a removable strand extending along the length of the prosthesis having a series of slip knots. The retention mechanism may also include a removable strand which engages overlying layers of a wrapped prosthesis by the passage of the strand through openings in the overlying layers. Manipulation of a user-accessible release strand permits release of the retention mechanism. The retention mechanism may also include a generally cylindrical sheath housing the vascular prosthesis, the sheath constructed to be split and removed to release the prosthesis. A method releases the prosthesis to expand at a target site using a retention mechanism.

### CROSS-REFERENCE TO OTHER APPLICATIONS

This application claims the benefit of U.S. provisional patent application No. 61/241,345 filed 10 Sep. 2009, the disclosure of which is incorporated by reference.

### BACKGROUND OF THE INVENTION

Today, there are a wide range of intravascular prostheses on the market for use in the treatment of aneurysms, stenosis, and other vascular disorders. Stents, stent grafts, and other vascular prostheses are well known for treating a myriad of diseases and illnesses in vasculature. For percutaneous interventions, many vascular prostheses are inserted into the body within a catheter and accurately and safely deployed at the desired treatment site.

Previously known self-expanding vascular prostheses can be retained in a catheter delivery configuration using an outer sheath; the prosthesis then self-expands when the outer sheath is retracted. See, for example, US patent application publication number US 2008/0021657 A1, assigned to the assignee of this application. Due to this configuration, several potentially undesirable effects are present during deployment of the prosthesis. Because the outer sheath is restraining the prosthesis, the frictional force between the prosthesis and outer sheath must be overcome to deploy the stent. The frictional force may be prohibitive to sheath withdrawal, and may shift the position of the prosthesis. Alternatively, self-expanding vascular prostheses can be secured to the outer surface of a delivery catheter; the prosthesis is then released from the delivery catheter at the target site within the patient. See, for example, U.S. Pat. Nos. 5,772,668 and 6,514,285.

This application is directed to systems in which self expanding vascular prosthesis are retained in their contracted states through the use of an outer delivery sheath. Portions of the vascular prosthesis may be secured to an inner delivery catheter, as in US 2008/0021657 A1, or an inner delivery catheter may not be used.

It is typically desirable that the vascular prosthesis have a high outward acting force to improve in vivo performance. However, this high outward acting force can result in a high frictional force during deployment, and requires the outer sheath, sometimes called the outer delivery sheath, to be strong both radially and longitudinally. A high deployment force is undesirable from safety, ergonomic, and control perspectives, e.g. placement accuracy. A high deployment force requires the use of stronger materials and/or a thicker outer sheath. These material and dimensional constraints are undesirable; the stronger materials are often more expensive and less flexible than traditional materials, and a thicker outer sheath moreover results in a larger device profile. Additionally, with a high deployment force, the outer sheath is more likely to stretch and neck down, resulting in additional deployment difficulties.

The vascular prosthesis is generally restrained in the outer sheath from the time the vascular prosthesis is loaded, packaged, sterilized, transported, and then deployed by the end-user. The device must remain operational following exposure to all of these environments, which can vary dramatically in temperature, humidity, and mechanical impact. Throughout these different environments, the self-expanding vascular prosthesis maintains a residual outward acting force. The changes in humidity and temperature can cause changes in the dimensions and physical properties of the device, resulting in undesirable deployment characteristics of the device. For example, sterilization through the use of ethylene oxide gas is a common sterilization procedure that requires elevated temperatures and high humidity to adequately sterilize the device. These conditions may cause the materials used in the device to expand and weaken, allowing the vascular prosthesis to expand radially and embed into the outer sheath, resulting in higher deployment forces and potential increases in profile. Additionally, the prosthesis material may have material properties such that elevated temperature results in the vascular prosthesis exerting a higher outward force against the outer sheath causing a further likelihood of higher deployment forces.

### BRIEF SUMMARY OF THE INVENTION

An example of a vascular prosthesis assembly includes a self expanding vascular prosthesis placeable in contracted and expanded states. The vascular prosthesis has distal and proximal ends and a generally cylindrical outer surface. The vascular prosthesis defines an axis and an axial length. A selectively releasable retention mechanism along the outer surface maintains the vascular prosthesis in the contracted state. The retention mechanism includes a strand extending along the length of the vascular prosthesis. The strand has a proximal end extending proximally from the vascular prosthesis for manipulation by a user. The strand extends around the outer surface at a plurality of release knot positions along the axis. The strand forms release knots at the release knot positions to maintain the vascular prosthesis in the contracted state. The retention mechanism also includes a release strand having a user-accessible proximal end, the release strand being coupled to the strand. The release knots are remotely releasable by user manipulation of the release strand to permit the vascular prosthesis to assume the expanded state. In some examples, the vascular prosthesis comprises of a wrapped stent with a series of outer apices; spaced apart groups of the release knots may be located at the outer apices.

Another example of a vascular prosthesis assembly includes a self expanding vascular prosthesis having a body placeable in contracted and expanded states. The vascular prosthesis has distal and proximal ends and a generally cylindrical outer surface. The vascular prosthesis defines an axis and an axial length. The body of the vascular prosthesis has openings formed therein. The body also has overlapping first and second body layers when in the contracted state. A selectively releasable retention mechanism along the outer surface maintains the vascular prosthesis in the contracted state. The retention mechanism includes one or more strands extending along the axial length. The one or more strands engage the first and second body layers by the passage of the at least one of the one or more strands through the openings in the body of the vascular prosthesis. The one or more strands include a user-accessible release strand to permit release of the retention mechanism by manipulation of the release strand.

Another example of a vascular prosthesis assembly includes a self expanding vascular prosthesis placeable in contracted and expanded states. The vascular prosthesis has distal and proximal ends and a generally cylindrical outer surface. The vascular prosthesis defines an axis and an axial length. A selectively releasable retention mechanism along the outer surface maintains the vascular prosthesis in the contracted state. The retention mechanism includes a generally cylindrical sheath housing the vascular prosthesis, the sheath having proximal and distal ends. The retention mechanism also includes means for selectively splitting the sheath at the distal end thereof. In some examples, the selectively releasable retention mechanism further comprises means for peeling the split distal end of the sheath back over the sheath towards the proximal end of the sheath. In some examples, one or more strands are used to assist in splitting the sheath. In some examples, one or more strands are embedded in the sheath to assist in splitting sheath.

An example of a delivery sheath is provided that reduces or eliminates embedding of the vascular prosthesis into the outer sheath. The example has a delivery sheath that becomes split starting at the distal end and is inverted to release the stent. In some examples, the delivery sheath comprises a sheath with embedded or loose strands included to aid in splitting or retraction. In some examples, the sheath consists of multiple layers to aid in manipulation.

A method for retaining and delivering a vascular prosthesis to a target site within a patient is carried out as follows. A self expanding vascular prosthesis placeable in contracted and expanded states is obtained. The vascular prosthesis has distal and proximal ends and a generally cylindrical outer surface. The vascular prosthesis defines an axis and an axial length. A selectively releasable retention mechanism is placed along the outer surface to maintain the vascular prosthesis in the contracted state. The placing step includes the following steps. A strand is secured along the radially contracted prosthesis through use of a plurality of release knots, the strand forming release knots at release knot positions to maintain the vascular prosthesis in the contracted state. The prosthesis is deployed by releasing the release knots to permit the vascular prosthesis to assume the expanded state. In some examples, the vascular prosthesis is placeable in the contracted state by wrapping, with discrete outer apices created in the contracted state; the release knot positions may coincide with the outer apices.

Alternative methods to retain the vascular prosthesis are provided, including the use of strands to maintain the compressed state of the vascular prosthesis. In some examples, the constraining strands are wire or suture. When constraining an alternating helical pattern, or serpentine pattern, by wrapping, a series of outer apices are the outermost layered elements. Constraining this series of outer apices at discrete locations allows the wrapped configuration to be held without an unwinding event. Examples are provided where each segment of the prosthesis is overlapped and constrained by the outermost layer. Additionally, examples are provided with features at each outer apex and/or the underlying layer designed to accept a strand.

Other features, aspects and advantages of the present invention can be seen on review of the drawings, the detailed description, and the claims which follow.

### BRIEF DESCRIPTION OF THE DRAWINGS

FIGS. 1 and 2 are side views of a vascular prosthesis having alternating helical portions shown in expanded and contracted states;

FIG. 3 illustrates a vascular prosthesis delivery system suitable for use with the vascular prosthesis of FIGS. 1 and 2;

FIG. 4 shows an atraumatic tip at the distal end of the delivery catheter of FIG. 3;

FIGS. 5 and 5A show an alternative embodiment of the vascular prosthesis of FIGS. 1 and 2 shown with the body in a flattened state and a contracted state, respectively;

FIG. 6 shows a vascular prosthesis delivery system of the type including a cartridge which houses the vascular prosthesis, the cartridge being mountable to an end of the outer delivery sheath;

FIG. 7 shows the outer delivery sheath of FIG. 6 after the vascular prosthesis has been placed into the interior of the outer delivery sheath and the cartridge has been removed;

FIG. 8 shows another example of the invention in which the vascular prosthesis has been placed within a smaller diameter storage region of an outer sheath for storage and sterilization;

FIG. 9 shows the outer delivery sheath of FIG. 8 with the vascular prosthesis moved from the storage region to the adjacent distal larger diameter region prior to delivery of the vascular prosthesis to the target site within the patient;

FIG. 10 shows a further example of the invention in which the outer delivery sheath has a tapering lumen and the prosthesis is within the smaller diameter proximal region for storage and sterilization;

FIG. 11 shows the outer delivery sheath from FIG. 10 with the vascular prosthesis moved to the distal larger diameter region prior to delivery of the vascular prosthesis to the target site within the patient; and

FIG. 12 shows a further example of the invention in which the outer delivery sheath has a tapering lumen in which the vascular prosthesis resides until delivery of the vascular prosthesis to the target site within the patient.

FIG. 13 shows an example of a vascular prosthesis delivery system with a retention mechanism in the form of a dual layer outer delivery sheath which can be pulled back for release of the vascular prosthesis.

FIG. 14 shows an example similar to that of FIG. 13 with a single layer outer delivery sheath.

FIG. 15 shows a single layer outer delivery sheath with strand support to aid peel back.

FIG. 16 shows an example of a vascular prosthesis delivery system using multiple fiber strands to selectively skive and peel the sheath.

FIG. 17 shows the example similar to that of FIG. 16 in which strands are laminated into the delivery sheath to skive and split open to sheath.

FIG. 18 illustrates an example of a vascular prosthesis delivery system in which multiple slip knots are tied around at the outer delivery sheath to maintain the vascular prosthesis in a contracted state, with an axially extending release strand used to untie or release the slip knots starting from the distal end.

FIG. 19 is a side view of vascular prosthesis delivery system in which spaced apart groups of slip knots are used to maintain the vascular prosthesis in the contracted state.

FIG. 20 is an enlarged view of a group of the slip knots of FIG. 19.

FIG. 21 shows a single strand of restraining material engaging overlying layers of the vascular prosthesis to maintain it in a contracted state.

FIG. 22 shows the use of two strands of retaining material, one looped through the overlying layers of the vascular prosthesis and the other passing through the loop, to maintain the vascular prosthesis in the contracted state.

FIG. 23 shows the use of three strands interwoven with each other and the vascular prosthesis to maintain the prosthesis in the contracted state.

FIG. 24 shows a strand engaging the stent in a slip knot fashion to maintain the prosthesis in the contracted state.

FIG. 25 shows an example of a single strand holding mating features of the vascular prosthesis at discrete locations.

FIG. 26 shows an example of two strands holding the vascular prosthesis at discrete locations.

FIG. 27 shows the proximal exit of strands which are used to activate the stent deployment.

### DETAILED DESCRIPTION OF THE INVENTION

The following description will typically be with reference to specific structural embodiments and methods. It is to be understood that there is no intention to limit the invention to the specifically disclosed embodiments and methods but that the invention may be practiced using other features, elements, methods and embodiments. Preferred embodiments are described to illustrate the present invention, not to limit its scope, which is defined by the claims. Those of ordinary skill in the art will recognize a variety of equivalent variations on the description that follows. Like elements in various embodiments are commonly referred to with like reference numerals.

One aspect of the present invention is the recognition of the drawbacks of previously known devices created by the vascular prosthesis exerting an outward radial force on the outer delivery sheath, discussed above, which causes embedding of the vascular prosthesis into the outer delivery sheath with the resultant increased and unpredictable delivery force. It would be desirable to provide an implantable vascular prosthesis delivery system with optimal delivery flexibility and profile, a low, predictable deployment force, and accurate vascular prosthesis placement.

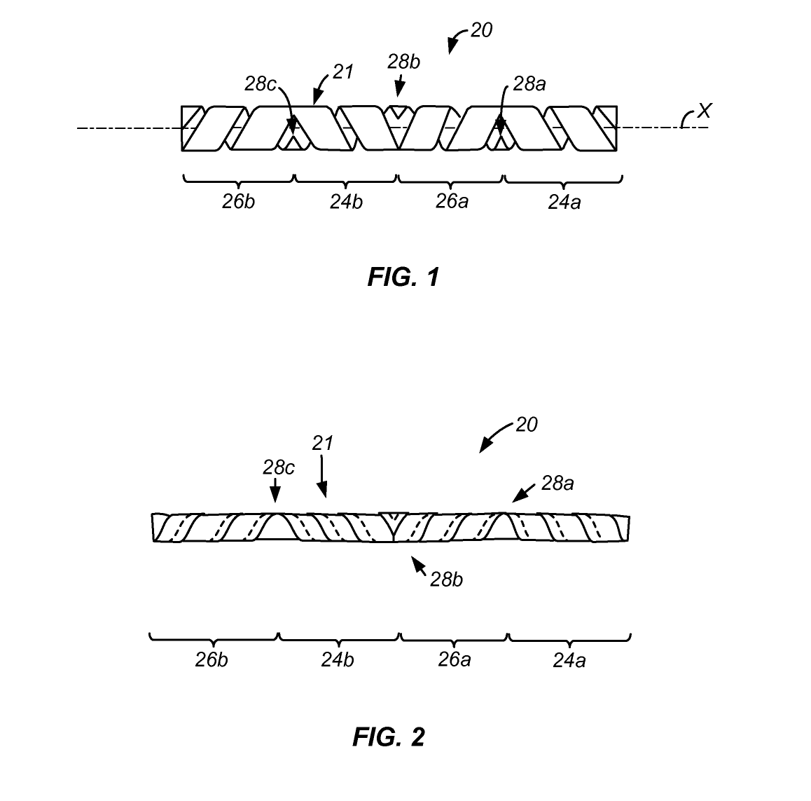

Referring now to FIGS. 1 and 2, a schematic representation of a vascular prosthesis 20 shown in an expanded, deployed state and a contracted, delivery state, respectively. Vascular prosthesis 20 is constructed from two or more helical portions having at least one change in the direction of rotation of the helices, and being joined at apex portions where the directions of rotation of adjacent helices change. In particular, first (i.e., proximal-most) helical portion 24a has a generally clockwise rotation about longitudinal axis X of prosthesis 20. Helical portion 26a adjoins the distal end of helical portion 24a at apex 28a and has a generally counter-clockwise rotation about longitudinal axis X. Helical portion 24b adjoins the distal end of helical portion 26a at apex 28b, and in turn is coupled to the proximal end of helical portion 26b at apex 28c. As a result of the alternating direction of rotation of the adjoining helical portions 24a, 26a, 24b and 26b of vascular prosthesis 20 includes three apices 28a, 28b and 28c that are oriented such that they point in alternating directions about the circumference of vascular prosthesis 20, generally in planes that are normal to longitudinal axis X of vascular prosthesis 20.

Alternating helical section 21 can be formed from a solid tubular member or sheet comprised of a shape memory material, such as nickel-titanium alloy (commonly known in the art as Nitinol). However, it should be appreciated that alternating helical section 21 may be constructed from any suitable material or processes recognized in the art. The prosthesis may then be laser cut or photoetched, using techniques that are known in the art, to define a specific pattern or geometry in the deployed configuration. Alternating helical section 21 can be cut or etched from the tube or sheet material so that helical portions 24a, 26a, 24b, 26b are integrally formed as a single monolithic body. However, it should be appreciated that separate helical portions may be mechanically coupled, such as by welding, soldering or installing mechanical fasteners to construct alternating helical section 21. An appropriate heat treatment then may be applied to alternating helical section 21 of vascular prosthesis 20 so that the device may be configured to self-deploy from a contracted delivery configuration to the expanded deployed configuration.

Referring now to FIG. 2, the vascular prosthesis 20 is shown in the contracted and partially overlapped, delivery configuration, wherein alternating helical section 21 is in the contracted, reduced diameter state. The vascular prosthesis 20 is placed in the contracted state by winding helical portions 24, 26 about longitudinal axis X. When vascular prosthesis 20 is loaded onto a delivery device, apices 28a and 28c are temporarily retained on an elongate body of a delivery system, and apex 28b and the distal and proximal ends of alternating helical section 21 are rotated relative to the elongate body until vascular prosthesis is in the contracted state as shown. As a result, apices 28a and 28c are wrapped radially inward of the remainder of vascular prosthesis 20 and will be generally referred to herein as “inner apices.” Conversely, apex 28b, which will be generally referred to as an “outer apex,” and the distal and proximal ends of alternating helical section 21 are wrapped radially outward of the remainder of alternating helical section 21.

Consequently, apices 28a and 28c are tightly wound onto the shaft of the delivery catheter and the remainder of each helical portion 24, 26 is wound against the shaft so that each turn of each portion 24, 26 slightly overlaps an adjacent turn. As a result, apex 28b and the distal and proximal ends of alternating helical section 21 are located furthest radially outward on the rolled alternating helical section 21 and are not secured to the delivery device. The overlap of the turns of helical portions 24, 26 is indicated by dashed lines in FIG. 2. The overlapping turns of alternating helical section 21 thus secure apices 28a and 28c when vascular prosthesis 20 is disposed within a delivery system. In addition, the overlapping of turns results in vascular prosthesis 20 having a unique deployment sequence that allows for increased control over its placement. Moreover, the unique configuration of alternating helical section 21 require a delivery system that allows for temporarily retaining the inner apices of alternating helical section 21 at least during loading.

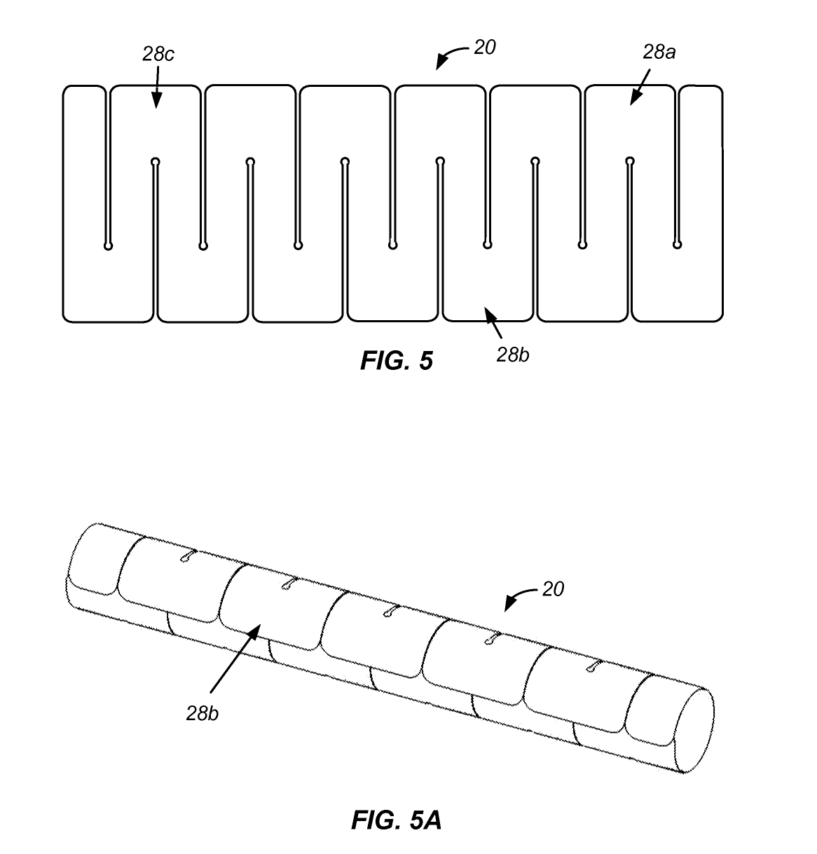

The present invention can be carried out with vascular prosthesis being constructed in a manner other than vascular prosthesis 20. For example, instead of being a ribbon-like material, the vascular prosthesis may be a wire having a round or other cross-sectional shape and may not have overlapping elements. Also, instead of having alternating helical sections, the entire prosthesis may be wound in a single direction. In another example, the vascular prosthesis is not helically wound but may be circumferentially wrapped; see FIGS. 5 and 5A. The prosthesis can alternatively be a radially compressible slotted tube design. In any event, the outer delivery sheath 42 maintains the vascular prosthesis 20 in the contracted state.

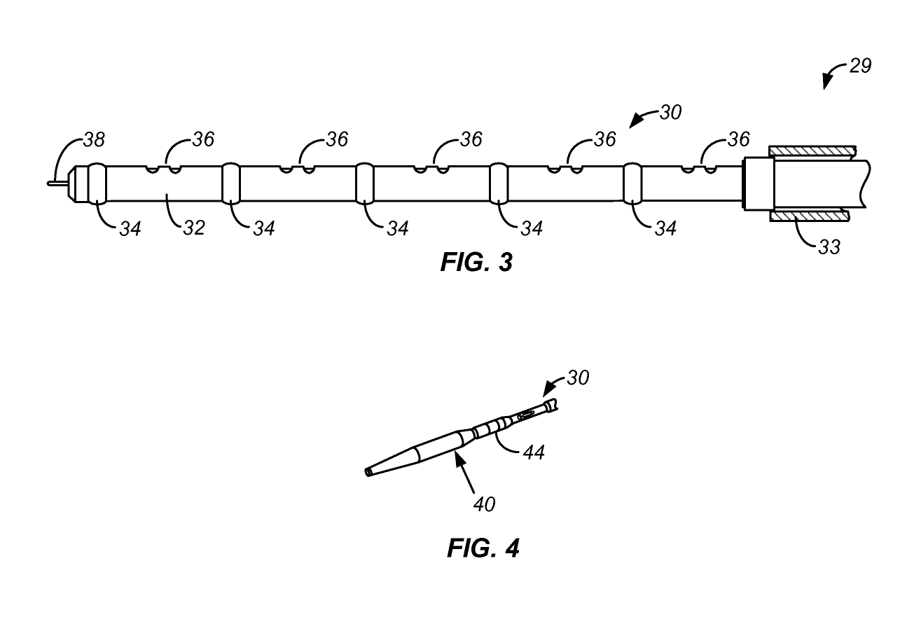

Referring to FIG. 3, one example of a delivery device 29 is shown. Delivery device 29 includes a delivery catheter 30, comprising an inner catheter body 32 and an outer delivery sheath 33 slideably mounted over the inner catheter body. Catheter 30 is the type shown in US patent application publication number US 2008/0021657 A1, the disclosure of which is incorporated by reference. The outer diameter of the inner catheter body 32 may be altered by pads (“bumps”) 34 that extend radially outward from the outer surface of catheter body 32. Pads 34 may be resilient or rigid rings that are coupled to the outer surface of catheter body 32 and spaced from retainers 36. The retainers are designed to hold the vascular prosthesis 20 at apices along one side of the prosthesis (“inner apices”), allowing the prosthesis to be held while the prosthesis is wrapped about the catheter body. The pads 34 may alternatively be designed with a geometry that mates with cavities in the constrained for deployment stent configuration. The catheter body 32 can be constructed from a high-strength resilient material, such as nylon, polyimide or polyetheretherketone (PEEK), so that it is flexible yet durable. The body may further be supported by a metallic matrix such as a braid or coil. Pads 34 may be made from a rigid or resilient material. Alternatively, the pads may be expanded from the inner shaft catheter body material, as one would blow a balloon. Additionally, marker bands to aid in stent position identification may be entrapped during the tip creation by placing them onto the inner shaft catheter prior to the blowing process.

Retainers 36 may be eyelets, notches, or similar structures in catheter body 32. A retaining wire, not shown, may be used to hold the prosthesis 20 to the catheter body 32. The retaining wire may be of a material such as high-strength polymer or Nitinol metallic wire. The retaining wire may run down the primary lumen of the catheter body 32 which may be sized to traverse over a guidewire 38. Alternatively, the retaining wire may be placed in a secondary, small diameter lumen.

Referring to FIG. 4, the inner catheter body 32 preferably includes an atraumatic tip 40 (not shown in FIG. 3), providing a smooth transition to the prosthesis 20. The tip 40 may comprise of a soft, lubricious material including, but not limited to polyether block amide (Pebax), nylon, polytetrafluoroethylene (PTFE), or fluorinated ethylene propylene (FEP). The atraumatic tip 40 may be comprised of a separate component attached to the inner catheter body 32, or the tip may be expanded from the inner catheter body material, as one would blow a balloon. Forming the tip 40 completely from the inner catheter body material reduces or potentially eliminates the concern of a tip breaking off and becoming an embolic risk. Material flow pre or post-blowing may be performed to create the desired tip transition, such as pre-necking the inner shaft material and blowing the entire tip configuration. Additionally, marker bands 44 may be entrapped during the tip creation by placing them onto the inner catheter body prior to the blowing process.

The following deployment mechanisms described apply to any self-expanding prosthesis configuration. The prosthesis may comprise a super-elastic material, such as Nitinol, or any suitable material recognized in the art, including polymers and biodegradable materials. The prosthesis design may consist of an alternating helix pattern as described above, such as a serpentine pattern as depicted in FIG. 5 with circumferential elements connected on alternating ends, or any other self-expanding design. Additionally, these mechanisms may be used with radially self expanding vascular prostheses for which balloon-expansion is used to provide additional deployment force for the vascular prosthesis.

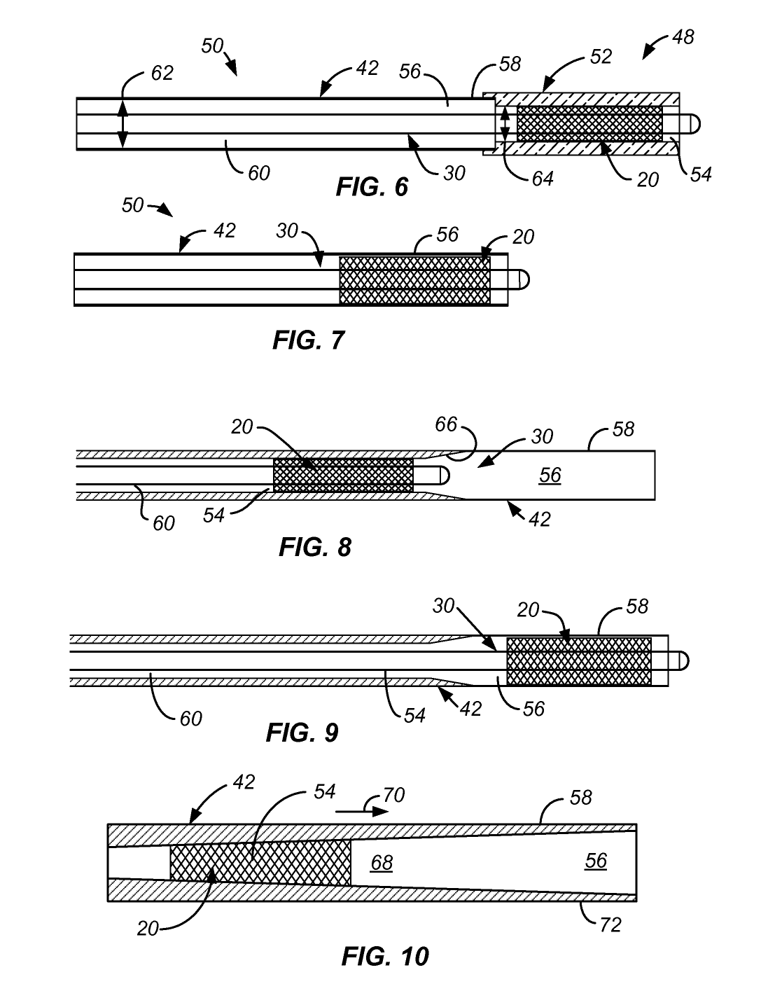

A first example of the invention will be discussed with reference to FIGS. 6 and 7. A delivery system 48 comprises a catheter assembly 50 and a cartridge 52. Catheter assembly 50 comprises an outer delivery sheath 42 and an inner delivery catheter 30. Cartridge 52 acts as an extension of outer delivery sheath 42. A vascular prosthesis 20 is mounted on a distal end of the inner delivery catheter 30. The cartridge 52 provides a temporary vascular prosthesis holding area 54. Vascular prosthesis 20 is typically housed within holding area 54 of cartridge 52 during sterilization and product storage. During clinical use, the prosthesis 20 is transferred from this storage region 54 into the final delivery region 56 in preparation for delivery to the patient site and implantation. This transfer takes place outside of the patient so that the extra force that may be required to transfer vascular prosthesis 20 from cartridge 52 into delivery region 56 of outer delivery sheath 42 is easily managed and does not create a threat of injury to the patient. After placement of vascular prosthesis 20 at delivery region 56, delivery sheath 42 is positioned at the target site within the patient. The short amount of time, typically a matter of minutes, between placement of vascular prosthesis 20 at delivery region 56 and removal of the vascular prosthesis from outer delivery sheath 42, eliminates the additional force that would otherwise be required to deploy the passenger prosthesis for at least two reasons. First, any embedding of the vascular prosthesis caused by plastic creep of the vascular prosthesis pressing against the outer delivery sheath is eliminated. Second, any embedding of the vascular prosthesis into the outer delivery sheath such as can result from sterilization or exposure to other environmental conditions would also be eliminated.

According to this example of this present invention, the vascular prosthesis 20 is captured inside of a constraining apparatus, cartridge 52, which can be separate from the catheter assembly. The vascular prosthesis 20 may be wrapped, then loaded into this temporary cartridge 52 that is sterilized separately from the rest of the device. Before clinical use and deployment, the cartridge 52 with the vascular prosthesis 20 loaded therein, is temporarily attached to the outer delivery sheath 42 and becomes an extension of outer delivery sheath 42. The cartridge 52 may be linked by friction fitting over the outer delivery sheath 42 of the catheter assembly, an o-ring feature, a clamshell design of the cartridge, the use of mating luers, or other appropriate connection mechanism. The cartridge 52 may be made from a lubricious material with sufficient strength to resist the prosthesis 20 from embedding into the inner surface of the cartridge during sterilization. Materials may include PTFE, FEP, polyimide-impregnated PTFE, Delrin®, polyethylene, Nitinol, or a composite such as a PTFE-lined braided tubing. As shown in FIG. 6, the cartridge 52 may be connected to the distal end 58 of the outer delivery sheath 42. Prior to device use, the vascular prosthesis 20 is transferred into a temporary holding area 54 of lumen 60 of outer delivery sheath 42 at the distal end 58 of the sheath to create a loaded catheter assembly 50 as shown in FIG. 7. The lumen 60 preferably has an internal diameter 62 equal to or greater than the cartridge internal diameter 64 of cartridge 52. A change in diameter of just 0.025 mm (0.001″) or 0.05 mm (0.002″) over the stent length is sufficient, but a change 0.076 mm (0.003″) or more is preferable. The cartridge 52 is then removed and the catheter assembly 50 is placed into the vessel. A pusher wire or alternate inner shaft may then be used to transfer the prosthesis 20 from the catheter assembly 50 into the treatment zone.

Alternatively, the cartridge 52 can be attached to the proximal end of the catheter assembly, not shown, and the prosthesis 20 can be transferred distally to its pre-delivery location using a pusher element. In this example, the lumen 60 of outer delivery sheath 42 also preferably has an internal diameter equal to or greater than the cartridge internal diameter. Again, a change in diameter of just 0.025 mm (0.001″) or 0.05 mm (0.002″) over the stent length is sufficient, but a change 0.076 mm (0.003″) or more is preferable. After transfer into the outer delivery sheath 42, the cartridge 52 is removed from the outer delivery sheath 42 and the loaded catheter assembly 50 is placed into the vessel. A pusher wire or alternate inner shaft may then be used to transfer the prosthesis along the catheter assembly into the treatment zone.

Cartridge 52 may be attached to the outer delivery sheath 42 during manufacturing. The cartridge 52 may be linked by a friction-fitting over the outer delivery sheath 42, an o-ring feature, a clamshell design of the cartridge, the use of mating luers, or an alternative mechanism. An inner delivery catheter 30 may be placed through both the outer delivery sheath 42 and the cartridge 52. The vascular prosthesis 20 may be loaded on the inner delivery catheter 30 and transferred into the cartridge 52. The entire system, including the outer delivery sheath 42, inner delivery catheter 30, the vascular prosthesis 20, and the cartridge 52, are then sterilized together or independently. At the clinical site, the vascular prosthesis 20 is transferred into the final sheath location 56 within outer delivery sheath 42 from the cartridge 52. If the cartridge 52 is attached to the proximal end of the outer delivery sheath 42, the vascular prosthesis 20 is pushed into or pulled through the lumen 60 of the outer delivery sheath and the cartridge 52 is removed. If the cartridge 52 is attached to the distal end 58 of the outer delivery sheath 42, the vascular prosthesis 20 may be pulled into the outer delivery sheath 42 from its proximal end using the delivery catheter 30. Alternatively, the vascular prosthesis 20 may be pushed into the outer delivery sheath 42 from the distal end 58 using a tool, such as a pusher wire, to advance the vascular prosthesis 20 through the cartridge 52.

In this example, vascular prosthesis 20 is initially secured to the delivery catheter 30 and can be released from the inner delivery catheter when the vascular prosthesis is outside of the outer delivery sheath 42. However, the invention can also be practiced when the vascular prosthesis 20 is not secured to an inner delivery catheter 30 so that it is pushed out of the distal end 58 of sheath 42 using other mechanisms, such as a pusher wire.

Another example of the invention relates to providing outer delivery sheath 42 with different internal diameters such that, as shown in FIGS. 8 and 9, the temporary vascular prosthesis storage region 54 has a smaller internal diameter than the vascular prosthesis delivery region 56, with regions 54, 56 connected by a tapered transition region 66. Instead of a smoothly tapering transition region 66, the transition region may have a series of smaller internal diameters reaching toward the proximal end. The vascular prosthesis 20 is shown in FIG. 8 constrained in the smaller diameter storage region 54 during sterilization and storage. While the entire vascular prosthesis 20 is shown in FIG. 8 to be located entirely proximal of the delivery region 56, in some examples, only a part of the prosthesis is proximal of delivery region 56. At the clinical site, prior to placement of the catheter assembly into the patient, the vascular prosthesis 20 is advanced into the larger diameter delivery region 56. Advancement of the vascular prosthesis prior to insertion of the delivery system into the patient, allows reduces forces caused by patient anatomical curvatures, accessory device interaction or elevated temperature effects. This allows for a lower deployment force within the vasculature compared to deployment without pre-advancement.

Differences in diameters between the storage region 54 and the delivery region 56 may be as little as 0.025 mm (0.001″) or 0.05 mm (0.002″), but preferably 0.076 mm (0.003″) or greater. The amount of the differences in diameters will depend at least in part upon the materials used, the forces exerted by vascular prosthesis 20 and the subsequent amount of embedding by vascular prosthesis 20 into outer delivery sheath 42. The thickness of stent 20 in the contracted state is preferably greater than the diameter change of the outer delivery sheath 42. This enables a pushing feature on the inner delivery catheter 30 at the proximal end of stent 20 to continuously contact the stent from the cartridge 52 or storage region 54 to the distal end of the delivery region 56. Contracted stent thickness may be achieved through individual wall thickness of stent 20 or the wrapping of stent 20 resulting in multiple layers. Alternatively, the stent 20 may be in intimate contact with the inner delivery catheter 30, e.g. through the use of a retaining wire.

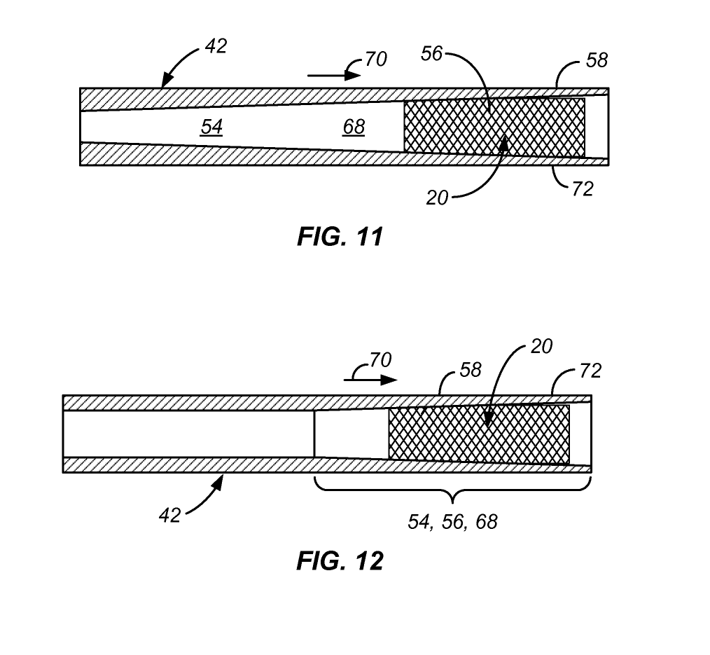

A further example of the invention will be discussed with reference to FIG. 10 and FIG. 11. In this example, the distal end 58 of outer delivery sheath 42 has an outwardly expanding, tapering lumen 68 when considered in a distal direction 70, that is toward the distal tip 72 of outer delivery sheath 42. This section may be a continuous taper, a taper over only a partial length of the stent, or include multiple, stepped diameters. During sterilization and storage, creep of the prosthesis and sheath due to the chronic outward force of the prosthesis, causes discreet lengths of each segment of the prosthesis to grow in diameter. When the prosthesis 20 is pushed through adjacent sections of a straight-profile sheath which have not experienced creep, deployment forces may be very high. Alternately, with the tapered sheath, the prosthesis 20 deploys in the distal direction 70, allowing each segment of the prosthesis to be pushed through a larger opening, thus reducing the forces of deployment. The reduction of deployment force occurs quite quickly after the initial movement of vascular prosthesis 20 in distal direction 70. Vascular prosthesis 20 can be stored and sterilized within proximal region 54 of tapering lumen 68. Vascular prosthesis 20 can then be advanced to the distal region 56 at the clinical site prior to placement of the catheter assembly into the patient. For a tapering lumen 68 having a length of 150 mm, a taper may be as little as 0.025 mm (0.001″) or 0.05 mm (0.002″), but preferably 0.076 mm (0.003″) or greater. This configuration acts to ease the deployment forces for an outer sheath pull-back mechanism. In another example, storage region 54 may be tapered as in FIGS. 10 and 11 but delivery region 56 may have a constant diameter; such constant diameter would typically be equal to or greater than the diameter of storage region 54 at the distal end of the storage region.

In an alternative example, shown in FIG. 12, tapering lumen 68 is relatively short and constitutes both the storage region 54 and the delivery region 56. That is, the storage and delivery regions at least substantially overlap and are therefore generally coextensive. The vascular prosthesis 20 is stored in the vascular prosthesis delivery region 56 through insertion of the delivery system to the patient's target site. Even if a certain amount of embedding had occurred, the taper of delivery region 56 causes the force necessary to push vascular prosthesis 20 out through the distal tip 72 of outer delivery sheath 42 to quickly drop after the initial movement of the vascular prosthesis. That is, after the initial movement of vascular prosthesis 20 distally through the coextensive storage/delivery region 54/56, the average diameter of vascular prosthesis 20 has increased to substantially immediately reduce the ejection force necessary. For a tapering delivery region 56 of this example having a length of slightly longer than the stent length, the overall taper along the length (diameter change) may be as little as 0.025 mm (0.001″) or 0.05 mm (0.002″), but preferably 0.076 mm (0.003″) or greater.

The sheath 42 may include, but is not limited to a metallic matrix of braid or coil, a PTFE liner, and a high-strength laminate layer. There are multiple methods of producing a tapered profile on the inner diameter of the sheath 42. The sheath may be laminated or stretched over a mandrel with the tapered outer diameter profile. The mandrel may be produced via multiple manufacturing methods including, but not limited to centerless grinding or Swiss screw machining. Additionally, stepped internal diameters may be incorporated with the tapered internal diameter. Therefore, tapering region 68 may include a single type of tapered segment or, for example, any combination of straight tapered segments, curved tapered segments and stepped tapered segments. The stepped tapered segments typically include generally axially directed surfaces and generally radially directed surfaces.

To further limit deployment force in the tapering delivery sheath concept exemplified in FIG. 11, the prosthesis 20 may be advanced just prior to device insertion into the patient. This allows the forces associated with prosthesis embedding into the outer shaft to be overcome when outside the patient, while the catheter is straight and at room temperature, when advancement forces will be lowest. As discussed above, such forces may arise as a result of sterilization, shelf life aging, or other changes to temperature and/or humidity. The same procedure may be used with the example of FIG. 12 in which the prosthesis is advanced a short distance through the coextensive storage/delivery region 54/56 just prior to device insertion into the patient in which the prosthesis is advanced a short distance through the coextensive storage/delivery region 54/56 just prior to device insertion into the patient.

The invention has been discussed in terms of smaller diameter storage regions and larger diameter delivery regions. In some examples, such as in FIGS. 9 and 10, the entire storage region will have a smaller diameter than any part of the delivery region. However, in some examples, there may be a portion of the storage region which has a diameter equal to or somewhat greater than a portion of the delivery region; even in such examples the average diameter of the storage region will be smaller than the average diameter of the delivery region so that the diameter of the storage region will be considered smaller than the diameter of the delivery region.

The examples of FIGS. 1-12 all use some type of outer delivery sheath 42 to constrain vascular prosthesis 20 with the vascular prosthesis being moved out of the delivery sheath for delivery at a target site within the patient. The examples of FIGS. 1-12 are the subject of the co-pending U.S. patent application Ser. No. 12/879,436 filed on the same day as this application and entitled Vascular Prosthesis Delivery System and Method, the disclosure of which is incorporated by reference.

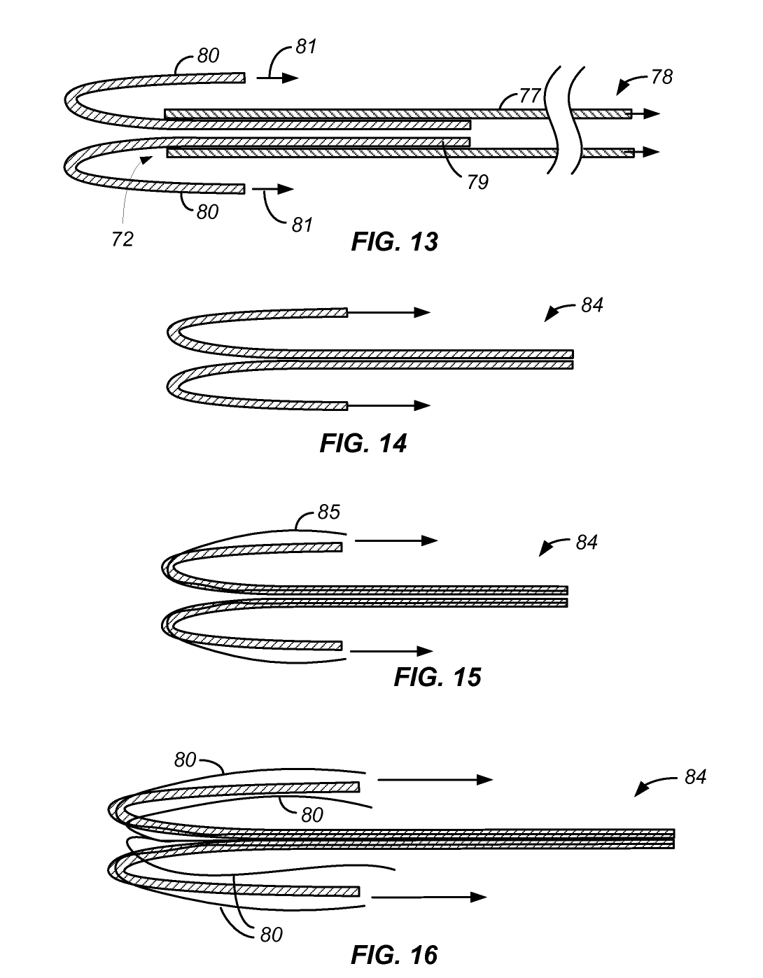

This invention relates to the following examples of apparatus and methods for a vascular prosthesis delivery system comprising various vascular prosthesis retention features, wherein the retention features can be controllably removed and can interface with, but are not limited to, features within and on the vascular prosthesis. FIGS. 13-16 illustrate apparatuses and methods for outer delivery sheaths that invaginate or peel-back to expose the underlying vascular prosthesis 20. In these deployment methods, the outer delivery sheath would be peeled back to deploy the prosthesis while the delivery catheter 30, not shown, and the attached prosthesis 20 are held stationary.

A double-sheath example is shown in FIG. 13, where the outer delivery sheath 78 includes an inner sheath 79, which is a ductile, lubricious, thin material, covered by a second, lubricious outer sheath 77, which supports the inner sheath in retaining the prosthesis, not shown in this figure, and provides mechanical integrity for forces required to access the deployment site. The outer sheath 77 may be conformal, or slit to ease the peel-back force. Peel back is initiated by pulling proximally on strands 80, indicated by arrows 81, which inverts the distal tip 72 of outer delivery sheath 78 causing sheath 78 to slit or separate, while pulling on the proximal end of outer delivery sheath 78 at the same time.

A single sheath peel-back design is shown in FIG. 14. The single delivery sheath 84 is a ductile, lubricious material capable of invaginating at low forces. The sheath material may include, but is not limited to a low durometer Pebax® polyether block amides, PTFE, or FEP. In the case when the material selection makes invagination challenging, fiber strands 85 or other similar materials may be used to skive delivery sheath 84 during peeling, as shown in FIG. 15. Alternatively, a scored or perforated material may be used to ease the peeling process, not shown.

In yet another example, shown in FIG. 16, multiple fiber strands 80 may be connected to the outer delivery sheath 84. In this case, two strands 80 skive the sheath 84, while two additional fibers peel-back the skived pieces of the sheath 84. The skiving strands 80 may include, but are not limited to high-strength suture material, metal wire, Kevlar, or other high-strength fibers. Strands 80 may be monofilament or multi-filament. Strands may be embedded in the polymer material of sheath 84 or arranged loosely within sheath 84. Peel-back may also be achieved by extending a delivery system element, not shown, and using materials such as metal, polymer tubing, or ribbon materials so that sheath 84 is controllably split. The vascular prosthesis delivery system may be configured to skive a specific sheath location prior to the sheath peel-back at this location. This sequence can reduce the forces required to peel away the delivery sheath 84. This can be accomplished by, for example, lengthening the element controlling the peel-back mechanism.

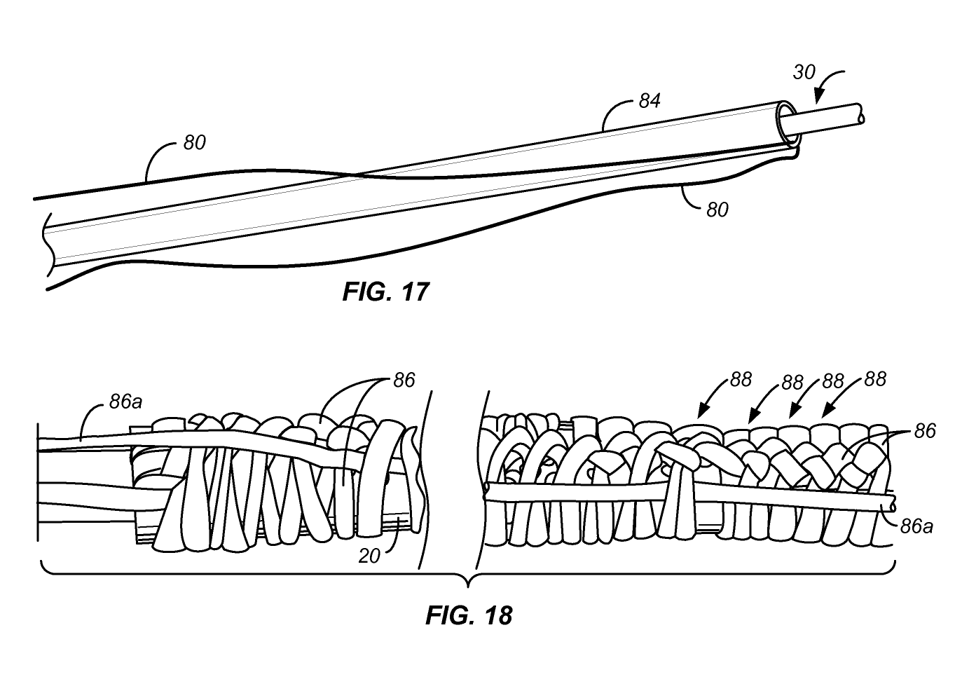

In the concept shown in FIG. 17, the constraining force from the outer delivery sheath 84 is relieved just prior to a sheath-pullback prosthesis deployment. The intent of the outer delivery sheath 84 is to restrain the prosthesis in its constrained state. However, during deployment, the restraining force exerted by the outer delivery sheath 84 on the vascular prosthesis can be quite high so to restrict the pull-back deployment of the system, resulting in high deployment forces and poor prosthesis placement accuracy.

FIG. 17 shows an example where two strands 80 are laminated into the material of delivery sheath 84. When placed under appropriate tension, the delivery sheath 84 is skived and split open. After splitting, the sheath 84 is removed using a conventional sheath-pullback mechanism. The skiving strands 80 may include, but are not limited to, high-strength suture material, metal wire, Kevlar, or other high-strength fibers. Alternatively, rather than requiring removal, the split sheath can be made of a material which may be left trapped against the vessel wall, using materials such as Dacron® polyester or ePTFE.

Other examples may use an additional inner or outer layer. The basic function of the skived layer is to provide the bulk of mechanical stability for constraining the prosthesis during sterilization and storage, and tracking the catheter to the deployment site. After one layer is split, the additional layer would loosely constrain the prosthesis against axially-retaining features and/or provide a low-friction surface to ease sheath pull-back. The additional constraining layer would ideally be a low-friction, thin material including, but not limited to, thin-walled FEP or PTFE. A temporary element, such as retaining wire, can be used to secure the prosthesis to the inner catheter body 30 to maintain the linear location of the prosthesis during outer delivery sheath removal. Following delivery catheter removal, the temporary element may be removed.

In some examples, a prosthesis is secured in a constrained state during prosthesis delivery using features on the prosthesis. These features can include, but are not limited to, braided or twisted fibers, metal wire of various thicknesses, geometries, and alloys, bioabsorbable or dissolvable bands, mechanical clasps, and/or marker bands. Included are common materials and configurations used in the production of surgical suture.

In one example, shown in FIG. 18, multi-filament or monofilament strands 86 of material, including fiber, suture, twine, or wire, which may be braided, twisted or not, can be positioned around the contracted vascular prosthesis 20 so that strands 86 constrain the prosthesis but do not pass through any features on the prosthesis, and remain on the surface of the prosthesis. These strands 86 typically include quick-release or slip knots or loops, which will be generically referred to as slip knots 88, acting as retention material 86 that allow for controllable prosthesis release when placed under tension in a specified direction, but constrain the prosthesis from opening in a radially-outward direction or unwinding. When placed under tension in one direction, typically by pulling on a release strand 86a, the retention material 86 is sequentially released in a distal to proximal direction so that the deployment of the prosthesis is controlled and so that the retention material is not apposed between the prosthesis 20 and the biological lumen. While FIG. 18 shows a single strand of material, a plurality of strands may be used.

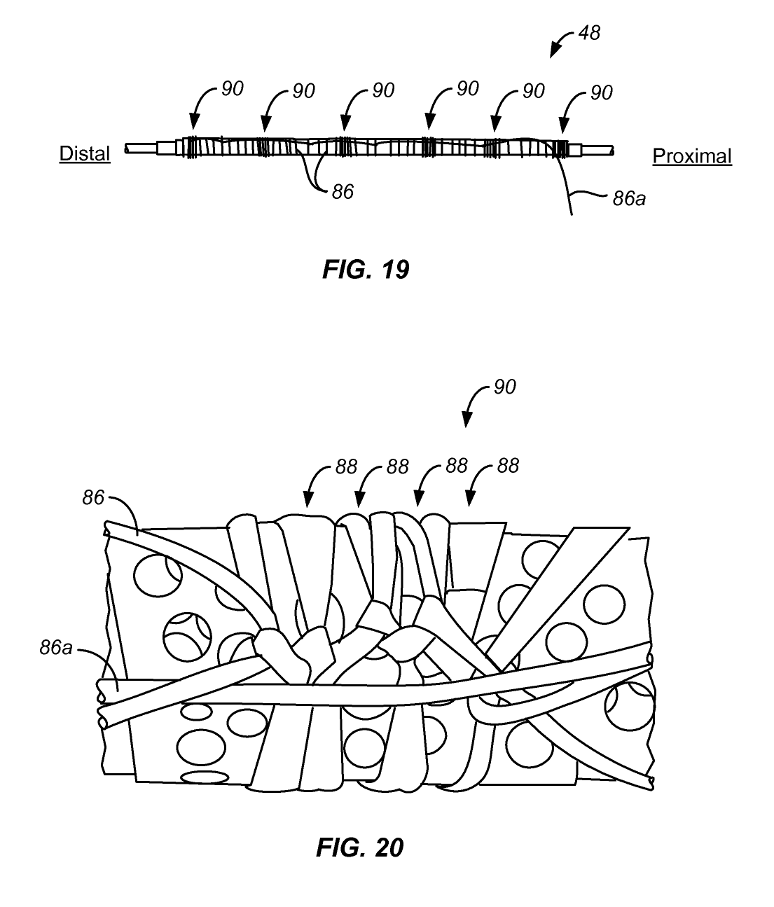

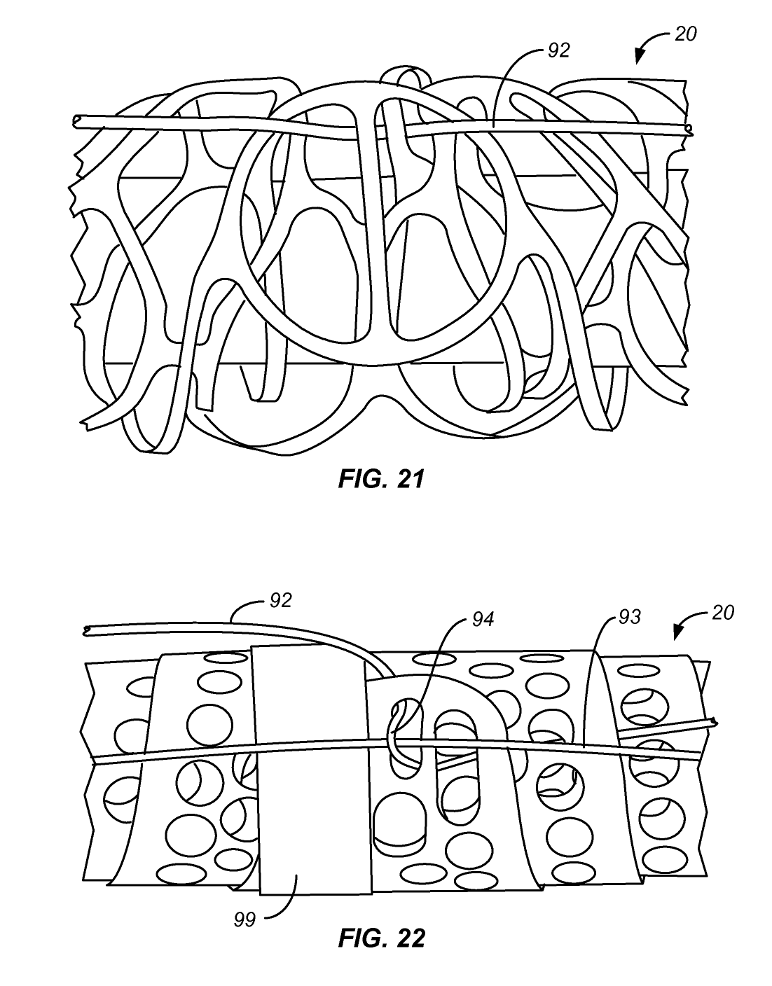

FIG. 18 illustrates use of closely spaced slip knots 88 along substantially the entire length of vascular prosthesis 20. FIGS. 19 and 20 illustrates an alternative embodiment in which groups 90 of slip knots 88 are spaced apart along the vascular prosthesis 20. It has been found that it is typically not necessary to use closely spaced slip knots 88 along the entire length of vascular prosthesis 20 but rather groups 90 of 4-6 slip knots 88 used at periodic intervals along vascular prosthesis 20 is typically sufficient. In one example, a vascular prosthesis having an axial length of 150 mm was restrained in its contracted state by the use of slip knots 88 in generally evenly spaced groups 90, each group 90 having approximately 5 to 15 slip knots 88. Each slip knots group 90 is centered approximately at each outer apex of the outer-most layer of wrapped stent.

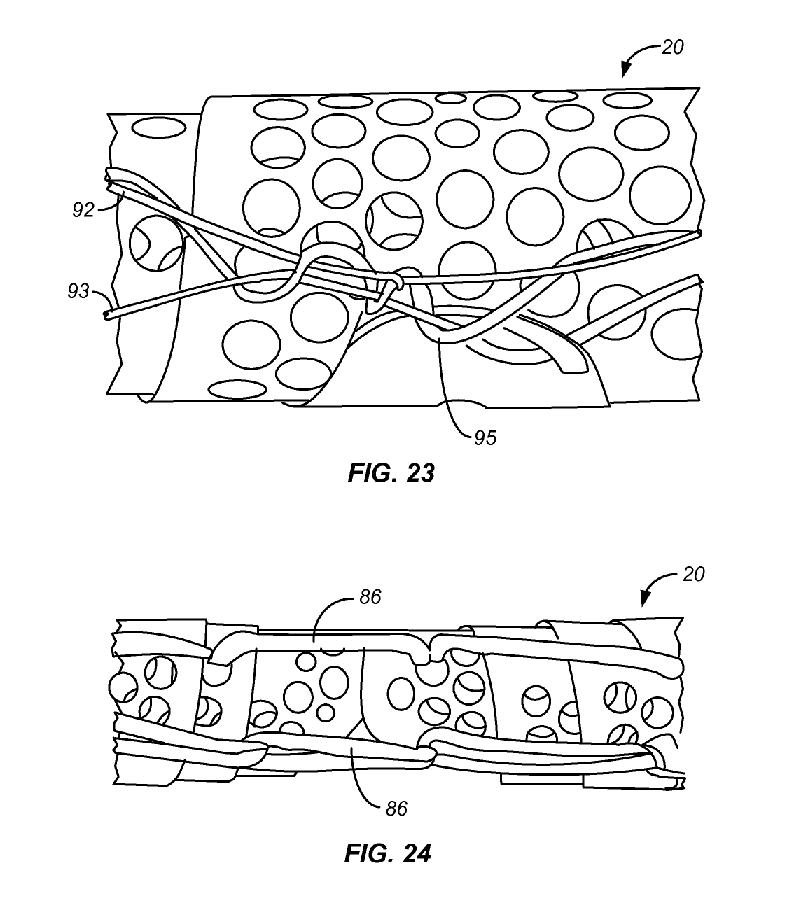

Strands 92 can be threaded through constrained vascular prosthesis 20 in various ways to restrain the prosthesis from opening. In one example, shown in FIG. 21, a single strand 92 of restraining material, possibly a metal wire, can be threaded through the vascular prosthesis 20 in a way to prevent the prosthesis from opening. The strand can secure an outer-most layer of the constrained stent to an underlying layer. In another example, shown in FIG. 22, two strands 92, 93, possibly metal wires, are used to retain prosthesis 20 in its contracted state. One strand 92 creates a series of loops 94 through two or more layers of constrained vascular prosthesis 20, while the second strand 93 is passed through the center of these loops, thereby preventing the vascular prosthesis from opening until the second strand is removed. The vascular prosthesis of FIG. 22 may be temporarily retained through the use of a retaining element, such as a marker band 99 as layers are connected. The retaining element is removed as a production step. In yet another example, shown in FIG. 23, two strands 92, 93, possibly metal wires, are threaded or looped through features on the vascular prosthesis 20, and the third strand 95, typically a monofilament thread, is used to bridge or thread the two other strands together, thus preventing the prosthesis from opening. Strand 95, followed by strands 92 and 93, is pulled to release the vascular prosthesis 20. In another example, shown in FIG. 24, strands 86, typically of a fiber material, are threaded through features of vascular prosthesis 20 in a slip knot design, thereby restraining layers of the vascular prosthesis in the contracted state. Pulling on the end of the strand 86 will therefore result in deployment. A combination of using slip knots 88 around vascular prosthesis 20 with securing the strand or strands through features on the prosthesis may also be used.

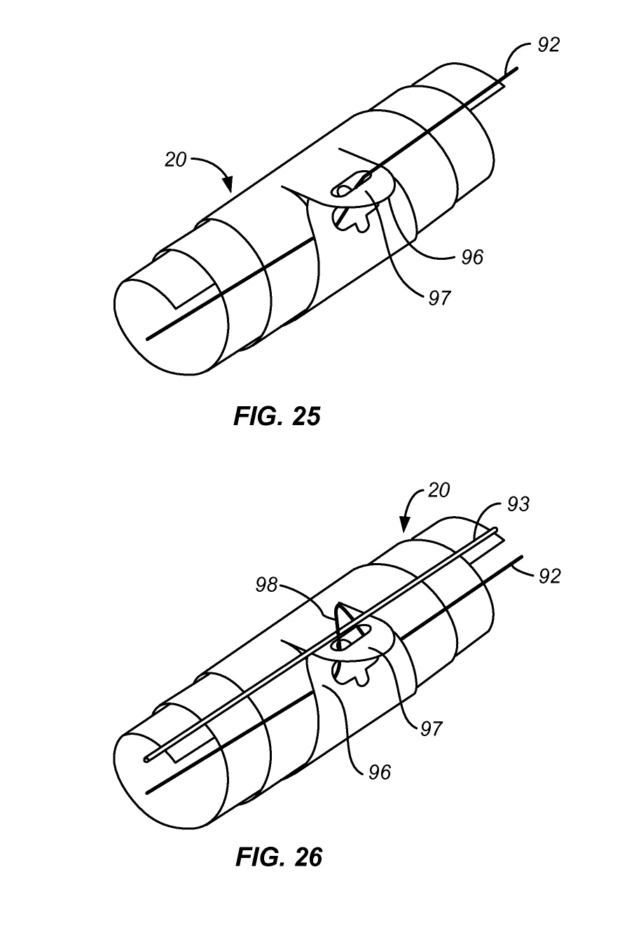

FIGS. 25 and 26 illustrates two additional ways of maintaining a vascular prosthesis 20 a contracted state. Vascular prosthesis 20 is of the type having one or more sets of apertured tabs 96, 97 which become aligned when in the contracted state. In FIG. 25 a single strand 92, typically, but not limited to, a metal wire, passes through a series of aligned openings in tabs 96, 97 to maintain vascular prosthesis 20 in the contracted state such as through wrapping. To deploy the prosthesis, strand 92 is withdrawn proximally to sequentially release the prosthesis at each set of aligned tabs 96, 97. Alternatively, deployment could be sequenced proximally to distally by pulling the strand 92 through the inner element around a reflection point distal of the prosthesis. Alternatively, strand 92 could be knotted in such a way as to release when tension is applied.

FIG. 26 shows the use of two strands 92, 93 typically both wires, with strand 92 creating a loop 98 passing through the apertures in a series of aligned aperture tabs 96, 97, with strand 93 passing through the exposed loop 98. To deploy the prosthesis 20, strand 93 would be withdrawn proximally to sequentially release each of the series of aperture tabs 96, 97. Alternatively, deployment could be sequenced proximally to distally by pulling the strand 93 through the inner element around a reflection point distal of the prosthesis. After deployment, the secondary strand 92 may be pulled out along with the delivery system. Alternatively, the strands 92, 93 could be knotted in such a way as to release when tension is applied. The dual strand configuration of FIG. 26 may have a lower deployment force when compared with the single strand configuration of FIG. 25.

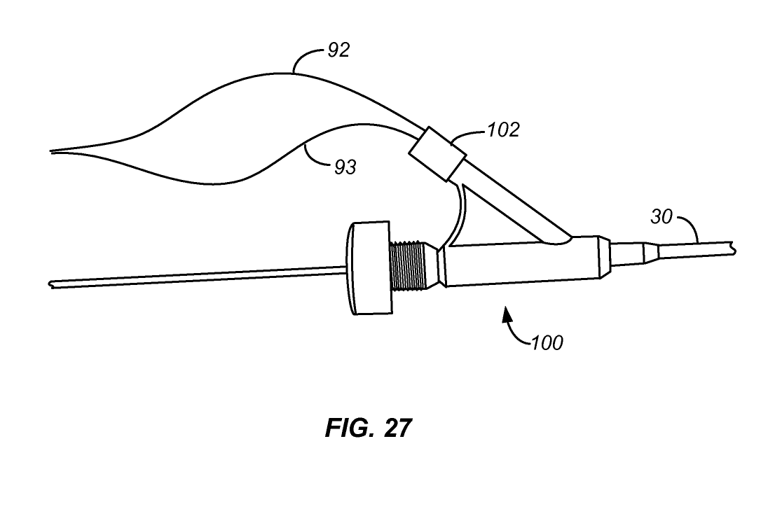

FIG. 27 shows a conventional proximal fitting 100 through which inner catheter body 30 passes. Fitting 100 includes a side port 102 shown with two strands, such as strands 92, 93, extending therefrom for manipulation by a user.

With the examples of FIGS. 21-26, vascular prosthesis 20 must have appropriate features to accept a constraining strand of retention material. When constraining an alternating helical pattern, or a serpentine pattern, a series of outer apices are typically at the outermost layered elements. Connecting this outermost element to an underlying layer at a single location allows the wrapped configuration to be held without an unwinding event. Features can be designed at each outer apex and/or the underlying layer to accept a constraining strand, such as a capture wire or suture. Features may include simple holes or strut configurations to create an opening in the two layers to accept a constraining strand. Optionally, the features on the underlying layer may be designed to pop-out from the surface to allow connection and even allow the wrapped vascular prosthesis to be further compressed radially when linked to the outer apex. Maintaining vascular prosthesis 20 in a contracted state using the retention material associated with examples of FIGS. 13-26 may offer advantages over a traditional retractable outer sheath. Some of those advantages may include a lower deployment force, smaller crossing profile, increased device flexibility, reduced device complexity and reduced cost.

An advantage of some examples is the reduction or elimination of any embedding of the vascular prosthesis into the outer sheath. The restraint systems may or may not be used in conjunction with other features designed to reduce deployment force and device property changes as a result of time, temperature, humidity, or other environmental factors. For example, a restraint system using a series of quick-release knots such as discussed with regard to FIGS. 18-20, can also be used with a traditional outer sheath. The suture material would restrain the vascular prosthesis during sterilization and shelf-life, and could be removed just before clinical insertion and use of the device. This would release the prosthesis, allowing the prosthesis to open up against the traditional outer sheath, removing any embedding or other effects that may occur as a result of sterilization or other environmental changes. In such use, the restraint systems can be removed in any direction and at an appropriate step of the procedure. For example, if the restraining features are removed before insertion into the patient, the features may be removed distally since this area of the device is accessible.

Another advantage of some examples is that they may allow for a combination of restraint and deployment mechanisms, such as to prevent any damage to the device or to a patient during device insertion, during travel to desired deployment location, and during deployment. For example, an outer sheath may cover the restraining system during device insertion, to protect from any mechanical harm the rough surface of the restraining system may cause.

In other examples, these securing features discussed with regard to FIGS. 21-26 can include holes specially designed and dimensioned for threading retention features in ways to improve manufacturing, safety, and releasing force and wear. Other features can include loops or tabs above the surface of the prosthesis that allow for reliable and consistent fastening of retention features. The securing features present on the prosthesis may maintain packing and compression of the prosthesis until desired deployment. The securing features can include, but not be limited to, loops, clasps, tabs, clips, threads, knots, bumps, ridges, eyelets, holes, and other features that allow for secure and reliable attachment to the prosthesis, as well as safe and controllable release of the prosthesis.

It is a further feature of some examples of the present invention to provide a deployment-assist handle that could safely, reliably, and controllably remove any retention features present on the vascular prosthesis. Such a handle could manipulate any features designed to release the vascular prosthesis restraints, either with or without force-assistance to the end-user. For a restraint system where a length of material or materials is pulled proximally, such as may occur with a series of slip knots discussed above with regard to FIGS. 18-20, the system could include a mechanism that would retract the material and spool or collect it into a compact area. This could be performed either by mechanical manipulation of the handle or through use of a motorized mechanism. For a restraint system where a retention features are broken or moved out of a restraint position, the device could consist of features that manipulate the retention features during deployment.

The above descriptions may have used terms such as above, below, top, bottom, over, under, et cetera. These terms may be used in the description and claims to aid understanding of the invention and not used in a limiting sense.

While the present invention is disclosed by reference to the preferred embodiments and examples detailed above, it is to be understood that these examples are intended in an illustrative rather than in a limiting sense. It is contemplated that modifications and combinations will occur to those skilled in the art, which modifications and combinations will be within the spirit of the invention and the scope of the following claims.

Any and all patents, patent applications and printed publications referred to above are incorporated by reference.

What is claimed is:

1. A vascular prosthesis assembly comprising:
a self expanding vascular prosthesis placeable in contracted and expanded states, the vascular prosthesis having distal and proximal ends and a generally cylindrical outer surface, the vascular prosthesis defining an axis and an axial length; and
a selectively releasable retention mechanism along the outer surface maintaining the vascular prosthesis in the contracted state, the retention mechanism comprising:
a strand extending along the length of the vascular prosthesis, the strand having a proximal end extending proximally from the vascular prosthesis for manipulation by a user;
the strand extending around the outer surface at a plurality of release knot positions along the axis, the strand forming release knots at the release knot positions to maintain the vascular prosthesis in the contracted state;
a release strand having a user-accessible proximal end, the release strand coupled to the strand; and
the release knots being remotely releasable by user manipulation of the release strand to permit the vascular prosthesis to assume the expanded state.

2. The assembly according to claim 1, wherein the vascular prosthesis comprises a wrapped stent with a series of outer apices.

3. The assembly according to claim 1, wherein the strand comprises at least one of:
monofilament materials; and
straight or braided or twisted multi-filament materials;
the materials being biologically compatible metal or nonmetal materials.

4. The assembly according claim 2, wherein the release knots comprise spaced apart groups of release knots.

5. The assembly according to claim 4, wherein the group of release knots are located at outer apices of the vascular prosthesis.

6. The assembly according to claim 1, wherein the release knots comprise a distal most release knot and the release strand extends from the distal most release knot.

7. The assembly according to claim 1, wherein the release strand and the strand comprises a continuous length of material.

8. The assembly according to claim 1, further comprising a plurality of said strands.

9. A vascular prosthesis assembly comprising:
a self expanding vascular prosthesis having a body placeable in contracted and expanded states, the vascular prosthesis having distal and proximal ends and a generally cylindrical outer surface, the vascular prosthesis defining an axis and an axial length;
the body of the vascular prosthesis having openings formed therein and overlapping first and second body layers when in the contracted state; and
a selectively releasable retention mechanism along the outer surface maintaining the vascular prosthesis in the contracted state, the retention mechanism comprising:
one or more strands extending along the axial length and engaging the first and second body layers by the passage of the at least one of the one or more strands through said openings;
the one or more strands comprising a user-accessible release strand to permit release of the retention mechanism by manipulation of the release strand.

10. The assembly according to claim 9, wherein the strand comprises at least one of:
monofilament materials; and
straight or braided or twisted multi-filament materials;
the materials being biologically compatible metal or nonmetal materials.

11. The assembly according to claim 9, wherein the release strand and the strand constitute a continuous length of material.

12. The assembly according to claim 9, further comprising a plurality of said strands.

13. A vascular prosthesis assembly comprising:
a self expanding vascular prosthesis placeable in contracted and expanded states, the vascular prosthesis having distal and proximal ends and a generally cylindrical outer surface, the vascular prosthesis defining an axis and an axial length; and
a selectively releasable retention mechanism along the outer surface maintaining the vascular prosthesis in the contracted state, the retention mechanism comprising:
a generally cylindrical sheath housing the vascular prosthesis and having proximal and distal ends; and
means for selectively splitting the sheath at the distal end thereof.

14. The assembly according to claim 13, wherein the selectively releasable retention mechanism further comprises means for peeling the split distal end of the sheath back over the sheath towards the proximal end of the sheath.

15. The assembly according to claim 13, wherein the sheath is pre-scored or perforated to ease splitting of the sheath.

16. The assembly according to claim 13, wherein one or more strands are used to assist in splitting the sheath.

17. The assembly according to claim 13, wherein one or more strands are embedded in the sheath to assist in splitting sheath.

18. A method for retaining and delivering a vascular prosthesis to a target site within a patient comprising:
obtaining a self expanding vascular prosthesis placeable in contracted and expanded states, the vascular prosthesis having distal and proximal ends and a generally cylindrical outer surface, the vascular prosthesis defining an axis and an axial length; and
placing a selectively releasable retention mechanism along the outer surface to maintain the vascular prosthesis in the contracted state, the placing step comprising:
securing a strand along the radially contracted prosthesis through use of a plurality of release knots, the strand forming release knots at release knot positions to maintain the vascular prosthesis in the contracted state; and
deploying prosthesis by releasing the release knots to permit the vascular prosthesis to assume the expanded state.

19. The method according to claim 18, wherein the vascular prosthesis is placeable in the contracted state by wrapping, with discrete outer apices created in the contracted state.

20. The method according to claim 19, wherein the release knot positions coincide with the discrete outer apices.
